SEQUENCE LISTING (1 sequence)

SEQ ID NO: 1 - protein, 9 aa, Homo sapiens
VMAPRTLFL

(19) United States
(12) Patent Application Publication
(10) Pub. No.: US 2020/0399377 A1
(43) Pub. Date: Dec. 24, 2020

(54) COMBINATION CANCER THERAPY WITH ANTI-CANCER AGENTS AND ANTIBODIES TARGETING A COMPLEX COMPRISING NON-CLASSICAL HLA-I AND NEOANTIGEN

(71) Applicant: AbeXXa Biologics Inc., Arlington, TX (US)

(72) Inventor: Jon Weidanz, Paradise, TX (US)

(21) Appl. No.: 16/975,364

(22) PCT Filed: Feb. 22, 2019
(86) PCT No.: PCT/US2019/019295

§ 371 Date: Aug. 24, 2020

Related U.S. Application Data
(60) Provisional application No. 62/634,522, filed on Feb. 23, 2018.

(51) Int. Cl. C07K 16/28; A61K 39/00; A61P 35/00; C07K 14/705; A61K 35/17

(52) U.S. Cl. CPC C07K 16/2833; A61K 39/001129; A61K 2039/507; C07K 14/70517; A61K 35/17; A61P 35/00

(57) ABSTRACT

Disclosed herein are methods and compositions for targeting a complex comprising a non-classical HLA-I and a neoantigen in cancer characterized by expression of CD94/NKG2A inhibitory receptor. Further disclosed herein are methods and compositions for combination cancer therapy.

### CROSS-REFERENCE

This patent application claims the benefit of U.S. Provisional Patent Application Ser. No. 62/634,522, filed Feb. 23, 2018, which is incorporated herein by reference in its entirety.

### SUMMARY OF THE DISCLOSURE

Disclosed herein, in some embodiments, are methods and compositions for targeting a complex comprising a non-classical HLA-I and a neoantigen in cancer characterized by expression of CD94/NKG2A inhibitory receptor. Further disclosed herein, in some embodiments, are methods and compositions for combination cancer therapy.

Disclosed herein, in certain embodiments, are methods of treating cancer characterized by expression of CD94/NKG2A inhibitory receptor in an individual in need thereof, comprising administering to the individual an antibody that selectively binds to a complex comprising a non-classical HLA-I and a neoantigen. In some instances, the methods further comprise administering an additional anti-cancer agent. In some instances, the methods further comprise assaying for expression of CD94/NKG2A inhibitory receptor in the individual. In some instances, the cancer is characterized by the overexpression of the CD94/NKG2A inhibitory receptor. In some instances, the antibody does not have a binding affinity to (i) the non-classical HLA-I alone; or (ii) the neoantigen alone. In some instances, the neoantigen comprises, consists essentially of, or consists of a sequence VMAPRTLFL. In some instances, the non-classical HLA-I is HLA-E, HLA-F, HLA-G, or HLA-H. In some instances, the non-classical HLA-I is HLA-E. In some instances, the HLA-E is HLA-E*0101 or HLA-E*0103. In some instances, the antibody selectively binds to the complex comprising the HLA-E and the neoantigen. In some instances, the antibody selectively binds to the complex comprising: (a) the HLA-E*0101 and the neoantigen; (b) the HLA-E*0103 and the neoantigen; or (c) the HLA-E*0101 and the neoantigen, and the HLA-E*0103 and the neoantigen. In some instances, the complex comprises the HLA-E and VMAPRTLFL. In some instances, the antibody is a murine antibody, a chimeric antibody, a camelid antibody, a humanized antibody, or a human antibody. In some instances, the antibody is a TCR-like antibody. In some instances, the antibody is a single domain antibody. In some instances, the single domain antibody is a camelid single domain antibody. In some instances, the antibody is a multispecific antibody. In some instances, the antibody is a multifunctional antibody. In some instances, the selective binding of the antibody to the complex comprising the non-classical HLA-I and the neoantigen inhibits the binding of the complex to the CD94/NKG2A inhibitory receptor. In some instances, the inhibition in binding of the complex to the CD94/NKG2A inhibitory receptor induces activation of natural killer (NK) cells. In some instances, the inhibition in binding of the complex to the CD94/NKG2A inhibitory receptor induces activation of CD8+ T cells. In some instances, the inhibition in binding of the complex to the CD94/NKG2A inhibitory receptor induces cell death of a cell expressing the non-classical HLA-I and the neoantigen. In some instances, the cell is a cancer cell. In some instances, the additional anti-cancer agent comprises CD20 inhibitor, HER-2 inhibitor, CD52 inhibitor, EGFR inhibitor, VEGF inhibitor, CCR4 inhibitor, PD-L1 inhibitor, SLAMF7 inhibitor, CD38 inhibitor, GD2 inhibitor, PTK-7 inhibitor, P-cadherin inhibitor, MCSF inhibitor, IDO1 inhibitor, CCR2 inhibitor, CXCR4 inhibitor, PD-1 inhibitor, CTLA-4 inhibitor, OX40 agonist, 4-1BB agonist, androgen receptor inhibitor, tyrosine kinase inhibitor, PARP inhibitor, chimeric antigen receptor T cells (CAR-T cells), oncolytic virus, and any combination thereof. In some instances, the additional anti-cancer agent comprises Rituximab, Trastuzumab, Alemutuzumab, Cetixumab, Bevacizumab, Panitumumab, Obinutuzumab, Mogamulizumab, Necitumumab, Atezolizumab, Elotuzumab, Daratumumab, Dinutuximab, and any combination thereof. In some instances, the antibody and the additional anti-cancer agent are administered concurrently. In some instances, the antibody and the additional anti-cancer agent are administered sequentially. In some instances, the antibody is administered prior to the additional anti-cancer agent. In some instances, the antibody is administered after the additional anti-cancer agent. In some instances, the antibody and the additional anti-cancer agent are administered in a unified dosage form. In some instances, the antibody and the additional anti-cancer agent are administered in a separate dosage form. In some instances, the antibody and the additional anti-cancer agent are administered continuously for 1, 2, 3, 4, 5, 6, 7, 8, 9, 10, 14, 15, 28, 30 or more days. In some instances, the antibody and the additional anti-cancer agent are administered at predetermined time intervals for 1, 2, 3, 4, 5, 6, 7, 8, 9, 10, 14, 15, 28, 30 or more days. In some instances, the antibody and the additional anti-cancer agent are administered intermittently for 1, 2, 3, 4, 5, 6, 7, 8, 9, 10, 14, 15, 28, 30 or more days. In some instances, the antibody and the additional anti-cancer agent are administered in 1 dose, 2 doses, 3 doses, 4 doses, 5 doses, 6 doses or more. In some instances, the antibody and the additional anti-cancer agent are administered at a therapeutically effective amount. In some instances, the cancer is breast cancer, kidney cancer, lung cancer, ovarian cancer, or colorectal cancer. In some instances, the cancer is a B-cell malignancy.

Disclosed herein, in some embodiments, are methods of treating cancer characterized by expression of CD94/NKG2A inhibitory receptor in an individual in need thereof, comprising administering to the individual an antibody that selectively binds to a complex comprising an HLA-E and a neoantigen. In some instances, the methods further comprise administering an additional anti-cancer agent. In some instances, the methods further comprise assaying for expression of CD94/NKG2A inhibitory receptor in the individual. In some instances, the cancer is characterized by the overexpression of the CD94/NKG2A inhibitory receptor. In some instances, the antibody does not have a binding affinity to (i) the HLA-E alone; or (ii) the neoantigen alone. In some instances, the neoantigen comprises, consists essentially of, or consists of a sequence VMAPRTLFL. In some instances, the HLA-E is HLA-E*0101 or HLA-E*0103. In some instances, the antibody selectively binds to the complex comprising: (a) the HLA-E*0101 and the neoantigen; (b) the HLA-E*0103 and the neoantigen; or (c) the HLA-E*0101 and the neoantigen, and the HLA-E*0103 and the neoantigen. In some instances, the complex comprises the HLA-E and VMAPRTLFL. In some instances, the antibody is a murine antibody, a chimeric antibody, a camelid antibody, a humanized antibody, or a human antibody. In some instances, the antibody is a TCR-like antibody. In some instances, the antibody is a single domain antibody. In some instances, the single domain antibody is a camelid single domain antibody. In some instances, the antibody is a multispecific antibody. In some instances, the antibody is a multifunctional antibody. In some instances, the selective binding of the antibody to the complex comprising the HLA-E and the neoantigen inhibits the binding of the complex to the CD94/NKG2A inhibitory receptor. In some instances, the inhibition in binding of the complex to the CD94/NKG2A inhibitory receptor induces activation of natural killer (NK) cells. In some instances, the inhibition in binding of the complex to the CD94/NKG2A inhibitory receptor induces activation of CD8+ T cells. In some instances, the inhibition in binding of the complex to the CD94/NKG2A inhibitory receptor induces cell death of a cell expressing the HLA-E and the neoantigen. In some instances, the cell is a cancer cell. In some instances, the additional anti-cancer agent comprises CD20 inhibitor, HER-2 inhibitor, CD52 inhibitor, EGFR inhibitor, VEGF inhibitor, CCR4 inhibitor, PD-L1 inhibitor, SLAMF7 inhibitor, CD38 inhibitor, GD2 inhibitor, PTK-7 inhibitor, P-cadherin inhibitor, MCSF inhibitor, IDO1 inhibitor, CCR2 inhibitor, CXCR4 inhibitor, PD-1 inhibitor, CTLA-4 inhibitor, OX40 agonist, 4-1BB agonist, androgen receptor inhibitor, tyrosine kinase inhibitor, PARP inhibitor, chimeric antigen receptor T cells (CAR-T cells), oncolytic virus, and any combination thereof. In some instances, the additional anti-cancer agent comprises Rituximab, Trastuzumab, Alemutuzumab, Cetixumab, Bevacizumab, Panitumumab, Obinutuzumab, Mogamulizumab, Necitumumab, Atezolizumab, Elotuzumab, Daratumumab, Dinutuximab, and any combination thereof. In some instances, the antibody and the additional anti-cancer agent are administered concurrently. In some instances, the antibody and the additional anti-cancer agent are administered sequentially. In some instances, the antibody is administered prior to the additional anti-cancer agent. In some instances, the antibody is administered after the additional anti-cancer agent. In some instances, the antibody and the additional anti-cancer agent are administered in a unified dosage form. In some instances, the antibody and the additional anti-cancer agent are administered in a separate dosage form. In some instances, the antibody and the additional anti-cancer agent are administered continuously for 1, 2, 3, 4, 5, 6, 7, 8, 9, 10, 14, 15, 28, 30 or more days. In some instances, the antibody and the additional anti-cancer agent are administered at predetermined time intervals for 1, 2, 3, 4, 5, 6, 7, 8, 9, 10, 14, 15, 28, 30 or more days. In some instances, the antibody and the additional anti-cancer agent are administered intermittently for 1, 2, 3, 4, 5, 6, 7, 8, 9, 10, 14, 15, 28, 30 or more days. In some instances, the antibody and the additional anti-cancer agent are administered in 1 dose, 2 doses, 3 doses, 4 doses, 5 doses, 6 doses or more. In some instances, the antibody and the additional anti-cancer agent are administered at a therapeutically effective amount. In some instances, the cancer is breast cancer, kidney cancer, lung cancer, ovarian cancer, or colorectal cancer. In some instances, the cancer is a B-cell malignancy.

Disclosed herein, in some embodiments, are methods of treating cancer in an individual in need thereof, comprising administering to the individual: (a) an antibody that selectively binds to a complex comprising a non-classical HLA-I and a neoantigen, and (b) an additional anti-cancer agent. In some instances, the antibody does not have a binding affinity to (i) the non-classical HLA-I alone; or (ii) the neoantigen alone. In some instances, the neoantigen comprises, consists essentially of, or consists of a sequence VMAPRTLFL. In some instances, the non-classical HLA-I is HLA-E, HLA-F, HLA-G, or HLA-H. In some instances, the non-classical HLA-I is HLA-E. In some instances, the HLA-E is HLA-E*0101 or HLA-E*0103. In some instances, the antibody selectively binds to the complex comprising the HLA-E and the neoantigen. In some instances, the antibody selectively binds to the complex comprising: (a) the HLA-E*0101 and the neoantigen; (b) the HLA-E*0103 and the neoantigen; or (c) the HLA-E*0101 and the neoantigen, and the HLA-E*0103 and the neoantigen. In some instances, the complex comprises the HLA-E and VMAPRTLFL. In some instances, the antibody is a murine antibody, a chimeric antibody, a camelid antibody, a humanized antibody, or a human antibody. In some instances, the antibody is a TCR-like antibody. In some instances, the antibody is a single domain antibody. In some instances, the single domain antibody is a camelid single domain antibody. In some instances, the antibody is a multispecific antibody. In some instances, the antibody is a multifunctional antibody. In some instances, the selective binding of the antibody to the complex comprising the non-classical HLA-I and the neoantigen inhibits the binding of the complex to a CD94/NKG2A inhibitory receptor. In some instances, the inhibition in binding of the complex to the CD94/NKG2A inhibitory receptor induces activation of natural killer (NK) cells. In some instances, the inhibition in binding of the complex to the CD94/NKG2A inhibitory receptor induces activation of CD8+ T cells. In some instances, the inhibition in binding of the complex to the CD94/NKG2A inhibitory receptor induces cell death of a cell expressing the non-classical HLA-I and the neoantigen. In some instances, the cell is a cancer cell. In some instances, the additional anti-cancer agent comprises CD20 inhibitor, HER-2 inhibitor, CD52 inhibitor, EGFR inhibitor, VEGF inhibitor, CCR4 inhibitor, PD-L1 inhibitor, SLAMF7 inhibitor, CD38 inhibitor, GD2 inhibitor, PTK-7 inhibitor, P-cadherin inhibitor, MCSF inhibitor, IDO1 inhibitor, CCR2 inhibitor, CXCR4 inhibitor, PD-1 inhibitor, CTLA-4 inhibitor, OX40 agonist, 4-1BB agonist, androgen receptor inhibitor, tyrosine kinase inhibitor, PARP inhibitor, chimeric antigen receptor T cells (CAR-T cells), oncolytic virus, and any combination thereof. In some instances, the additional anti-cancer agent comprises Rituximab, Trastuzumab, Alemutuzumab, Cetixumab, Bevacizumab, Panitumumab, Obinutuzumab, Mogamulizumab, Necitumumab, Atezolizumab, Elotuzumab, Daratumumab, Dinutuximab, and any combination thereof. In some instances, the antibody and the additional anti-cancer agent are administered concurrently. In some instances, the antibody and the additional anti-cancer agent are administered sequentially. In some instances, the antibody is administered prior to the additional anti-cancer agent. In some instances, the antibody is administered after the additional anti-cancer agent. In some instances, the antibody and the additional anti-cancer agent are administered in a unified dosage form. In some instances, the antibody and the additional anti-cancer agent are administered in a separate dosage form In some instances, the antibody and the additional anti-cancer agent are administered continuously for 1, 2, 3, 4, 5, 6, 7, 8, 9, 10, 14, 15, 28, 30 or more days. In some instances, the antibody and the additional anti-cancer agent are administered at predetermined time intervals for 1, 2, 3, 4, 5, 6, 7, 8, 9, 10, 14, 15, 28, 30 or more days. In some instances, the antibody and the additional anti-cancer agent are administered intermittently for 1, 2, 3, 4, 5, 6, 7, 8, 9, 10, 14, 15, 28, 30 or more days. In some instances, the antibody and the additional anti-cancer agent are administered in 1 dose, 2 doses, 3 doses, 4 doses, 5 doses, 6 doses or more. In some instances, the antibody and the additional anti-cancer agent are administered at a therapeutically effective amount. In some instances, the cancer is characterized by expression of the CD94/NKG2A inhibitory receptor. In some instances, the cancer is characterized by overexpression of the CD94/NKG2A inhibitory receptor. In some instances, the methods further comprise assaying for expression of the CD94/NKG2A inhibitory receptor in the individual. In some instances, the cancer is breast cancer, kidney cancer, lung cancer, ovarian cancer, or colorectal cancer. In some instances, the cancer is a B-cell malignancy.

Disclosed herein, in some embodiments, are methods of treating cancer in an individual in need thereof, comprising administering to the individual: (a) an antibody that selectively binds to a complex comprising a HLA-E and a neoantigen, and (b) an additional anti-cancer agent. In some instances, the antibody does not have a binding affinity to (i) the HLA-E alone; or (ii) the neoantigen alone. In some instances, the neoantigen comprises, consists essentially of, or consists of a sequence VMAPRTLFL. In some instances, the HLA-E is HLA-E*0101 or HLA-E*0103. In some instances, the antibody selectively binds to the complex comprising: (a) the HLA-E*0101 and the neoantigen; (b) the HLA-E*0103 and the neoantigen; or (c) the HLA-E*0101 and the neoantigen, and the HLA-E*0103 and the neoantigen. In some instances, the complex comprises the HLA-E and VMAPRTLFL. In some instances, the antibody is a murine antibody, a chimeric antibody, a camelid antibody, a humanized antibody, or a human antibody. In some instances, the antibody is a TCR-like antibody. In some instances, the antibody is a single domain antibody. In some instances, the single domain antibody is a camelid single domain antibody. In some instances, the antibody is a multispecific antibody. In some instances, the antibody is a multifunctional antibody. In some instances, the selective binding of the antibody to the complex comprising the HLA-E and the neoantigen inhibits the binding of the complex to a CD94/NKG2A inhibitory receptor. In some instances, the inhibition in binding of the complex to the CD94/NKG2A inhibitory receptor induces activation of natural killer (NK) cells. In some instances, the inhibition in binding of the complex to the CD94/NKG2A inhibitory receptor induces activation of CD8+ T cells. In some instances, the inhibition in binding of the complex to the CD94/NKG2A inhibitory receptor induces cell death of a cell expressing the HLA-E and the neoantigen. In some instances, the cell is a cancer cell. In some instances, the additional anti-cancer agent comprises CD20 inhibitor, HER-2 inhibitor, CD52 inhibitor, EGFR inhibitor, VEGF inhibitor, CCR4 inhibitor, PD-L1 inhibitor, SLAMF7 inhibitor, CD38 inhibitor, GD2 inhibitor, PTK-7 inhibitor, P-cadherin inhibitor, MCSF inhibitor, IDO1 inhibitor, CCR2 inhibitor, CXCR4 inhibitor, PD-1 inhibitor, CTLA-4 inhibitor, OX40 agonist, 4-1BB agonist, androgen receptor inhibitor, tyrosine kinase inhibitor, PARP inhibitor, chimeric antigen receptor T cells (CAR-T cells), oncolytic virus, and any combination thereof. In some instances, the additional anti-cancer agent comprises Rituximab, Trastuzumab, Alemutuzumab, Cetixumab, Bevacizumab, Panitumumab, Obinutuzumab, Mogamulizumab, Necitumumab, Atezolizumab, Elotuzumab, Daratumumab, Dinutuximab, and any combination thereof. In some instances, the antibody and the additional anti-cancer agent are administered concurrently. In some instances, the antibody and the additional anti-cancer agent are administered sequentially. In some instances, the antibody is administered prior to the additional anti-cancer agent. In some instances, the antibody is administered after the additional anti-cancer agent. In some instances, the antibody and the additional anti-cancer agent are administered in a unified dosage form. In some instances, the antibody and the additional anti-cancer agent are administered in a separate dosage form. In some instances, the antibody and the additional anti-cancer agent are administered continuously for 1, 2, 3, 4, 5, 6, 7, 8, 9, 10, 14, 15, 28, 30 or more days. In some instances, the antibody and the additional anti-cancer agent are administered at predetermined time intervals for 1, 2, 3, 4, 5, 6, 7, 8, 9, 10, 14, 15, 28, 30 or more days. In some instances, the antibody and the additional anti-cancer agent are administered intermittently for 1, 2, 3, 4, 5, 6, 7, 8, 9, 10, 14, 15, 28, 30 or more days. In some instances, the antibody and the additional anti-cancer agent are administered in 1 dose, 2 doses, 3 doses, 4 doses, 5 doses, 6 doses or more. In some instances, the antibody and the additional anti-cancer agent are administered at a therapeutically effective amount. In some instances, the cancer is characterized by expression of the CD94/NKG2A inhibitory receptor. In some instances, the cancer is characterized by overexpression of the CD94/NKG2A inhibitory receptor. In some instances, the methods further comprise assaying for expression of the CD94/NKG2A inhibitory receptor in the individual. In some instances, the cancer is breast cancer, kidney cancer, lung cancer, ovarian cancer, or colorectal cancer. In some instances, the cancer is a B-cell malignancy.

Disclosed herein, in some embodiments, are methods of inducing cell death of a cancer cell in a tumor microenvironment characterized by expression of CD94/NKG2A inhibitory receptor in an individual in need thereof, comprising administering to the individual an antibody that selectively binds to a complex comprising a non-classical HLA-I and a neoantigen, wherein the complex is expressed by the cancer cell. In some instances, the methods further comprise administering an additional anti-cancer agent. In some instances, the methods further comprise assaying for expression of the CD94/NKG2A inhibitory receptor in the individual. In some instances, the tumor microenvironment is characterized by overexpression of the CD94/NKG2A inhibitory receptor. In some instances, the antibody does not have a binding affinity to (i) the non-classical HLA-I alone; or (ii) the neoantigen alone. In some instances, the neoantigen comprises, consists essentially of, or consists of a sequence VMAPRTLFL. In some instances, the non-classical HLA-I is HLA-E, HLA-F, HLA-G, or HLA-H. In some instances, the non-classical HLA-I is HLA-E. In some instances, the HLA-E is HLA-E*0101 or HLA-E*0103. In some instances, the antibody selectively binds to the complex comprising the HLA-E and the neoantigen. In some instances, the antibody selectively binds to the complex comprising: (a) the HLA-E*0101 and the neoantigen; (b) the HLA-E*0103 and the neoantigen; or (c) the HLA-E*0101 and the neoantigen, and the HLA-E*0103 and the neoantigen. In some instances, the complex comprises the HLA-E and VMAPRTLFL. In some instances, the antibody is a murine antibody, a chimeric antibody, a camelid antibody, a humanized antibody, or a human antibody. In some instances, the antibody is a TCR-like antibody. In some instances, the antibody is a single domain antibody. In some instances, the single domain antibody is a camelid single domain antibody. In some instances, the antibody is a multispecific antibody. In some instances, the antibody is a multifunctional antibody. In some instances, the selective binding of the antibody to the complex comprising the non-classical HLA-I and the neoantigen inhibits the binding of the complex to the CD94/NKG2A inhibitory receptor. In some instances, the inhibition in binding of the complex to the CD94/NKG2A inhibitory receptor induces activation of natural killer (NK) cells. In some instances, the inhibition in binding of the complex to the CD94/NKG2A inhibitory receptor induces activation of CD8+ T cells. In some instances, the additional anti-cancer agent comprises CD20 inhibitor, HER-2 inhibitor, CD52 inhibitor, EGFR inhibitor, VEGF inhibitor, CCR4 inhibitor, PD-L1 inhibitor, SLAMF7 inhibitor, CD38 inhibitor, GD2 inhibitor, PTK-7 inhibitor, P-cadherin inhibitor, MCSF inhibitor, IDO1 inhibitor, CCR2 inhibitor, CXCR4 inhibitor, PD-1 inhibitor, CTLA-4 inhibitor, OX40 agonist, 4-1BB agonist, androgen receptor inhibitor, tyrosine kinase inhibitor, PARP inhibitor, chimeric antigen receptor T cells (CAR-T cells), oncolytic virus, and any combination thereof. In some instances, the additional anti-cancer agent comprises Rituximab, Trastuzumab, Alemutuzumab, Cetixumab, Bevacizumab, Panitumumab, Obinutuzumab, Mogamulizumab, Necitumumab, Atezolizumab, Elotuzumab, Daratumumab, Dinutuximab, and any combination thereof. In some instances, the antibody and the additional anti-cancer agent are administered concurrently. In some instances, the antibody and the additional anti-cancer agent are administered sequentially. In some instances, the antibody is administered prior to the additional anti-cancer agent. In some instances, the antibody is administered after the additional anti-cancer agent. In some instances, the antibody and the additional anti-cancer agent are administered in a unified dosage form. In some instances, the antibody and the additional anti-cancer agent are administered in a separate dosage form. In some instances, the antibody and the additional anti-cancer agent are administered continuously for 1, 2, 3, 4, 5, 6, 7, 8, 9, 10, 14, 15, 28, 30 or more days. In some instances, the antibody and the additional anti-cancer agent are administered at predetermined time intervals for 1, 2, 3, 4, 5, 6, 7, 8, 9, 10, 14, 15, 28, 30 or more days. In some instances, the antibody and the additional anti-cancer agent are administered intermittently for 1, 2, 3, 4, 5, 6, 7, 8, 9, 10, 14, 15, 28, 30 or more days. In some instances, the antibody and the additional anti-cancer agent are administered in 1 dose, 2 doses, 3 doses, 4 doses, 5 doses, 6 doses or more. In some instances, the antibody and the additional anti-cancer agent are administered at a therapeutically effective amount. In some instances, the cancer cell is a breast cancer cell, a kidney cancer cell, a lung cancer cell, an ovarian cancer cell, or a colorectal cancer cell. In some instances, the cancer cell is a malignant B cell.

Disclosed herein, in some embodiments, are methods of inducing cell death of a cancer cell in a tumor microenvironment characterized by expression of CD94/NKG2A inhibitory receptor in an individual in need thereof, comprising administering to the individual an antibody that selectively binds to a complex comprising an HLA-E and a neoantigen, wherein the complex is expressed by the cancer cell. In some instances, the methods further comprise administering an additional anti-cancer agent. In some instances, the methods further comprise assaying for expression of the CD94/NKG2A inhibitory receptor in the individual. In some instances, the tumor microenvironment is characterized by the overexpression of the CD94/NKG2A inhibitory receptor. In some instances, the antibody does not have a binding affinity to (i) the HLA-E alone; or (ii) the neoantigen alone. In some instances, the neoantigen comprises, consists essentially of, or consists of a sequence VMAPRTLFL. In some instances, the HLA-E is HLA-E*0101 or HLA-E*0103. In some instances, the antibody selectively binds to the complex comprising: (a) the HLA-E*0101 and the neoantigen; (b) the HLA-E*0103 and the neoantigen; or (c) the HLA-E*0101 and the neoantigen, and the HLA-E*0103 and the neoantigen. In some instances, the complex comprises the HLA-E and VMAPRTLFL. In some instances, the antibody is a murine antibody, a chimeric antibody, a camelid antibody, a humanized antibody, or a human antibody. In some instances, the antibody is a TCR-like antibody. In some instances, the antibody is a single domain antibody. In some instances, the single domain antibody is a camelid single domain antibody. In some instances, the antibody is a multispecific antibody. In some instances, the antibody is a multifunctional antibody. In some instances, the selective binding of the antibody to the complex comprising the HLA-E and the neoantigen inhibits the binding of the complex to the CD94/NKG2A inhibitory receptor. In some instances, the inhibition in binding of the complex to the CD94/NKG2A inhibitory receptor induces activation of natural killer (NK) cells. In some instances, the inhibition in binding of the complex to the CD94/NKG2A inhibitory receptor induces activation of CD8+ T cells. In some instances, the additional anti-cancer agent comprises CD20 inhibitor, HER-2 inhibitor, CD52 inhibitor, EGFR inhibitor, VEGF inhibitor, CCR4 inhibitor, PD-L1 inhibitor, SLAMF7 inhibitor, CD38 inhibitor, GD2 inhibitor, PTK-7 inhibitor, P-cadherin inhibitor, MCSF inhibitor, IDO1 inhibitor, CCR2 inhibitor, CXCR4 inhibitor, PD-1 inhibitor, CTLA-4 inhibitor, OX40 agonist, 4-1BB agonist, androgen receptor inhibitor, tyrosine kinase inhibitor, PARP inhibitor, chimeric antigen receptor T cells (CAR-T cells), oncolytic virus, and any combination thereof. In some instances, the additional anti-cancer agent comprises Rituximab, Trastuzumab, Alemutuzumab, Cetixumab, Bevacizumab, Panitumumab, Obinutuzumab, Mogamulizumab, Necitumumab, Atezolizumab, Elotuzumab, Daratumumab, Dinutuximab, and any combination thereof. In some instances, the antibody and the additional anti-cancer agent are administered concurrently. In some instances, the antibody and the additional anti-cancer agent are administered sequentially. In some instances, the antibody is administered prior to the additional anti-cancer agent. In some instances, the antibody is administered after the additional anti-cancer agent. In some instances, the antibody and the additional anti-cancer agent are administered in a unified dosage form. In some instances, the antibody and the additional anti-cancer agent are administered in a separate dosage form. In some instances, the antibody and the additional anti-cancer agent are administered continuously for 1, 2, 3, 4, 5, 6, 7, 8, 9, 10, 14, 15, 28, 30 or more days. In some instances, the antibody and the additional anti-cancer agent are administered at predetermined time intervals for 1, 2, 3, 4, 5, 6, 7, 8, 9, 10, 14, 15, 28, 30 or more days. In some instances, the antibody and the additional anti-cancer agent are administered intermittently for 1, 2, 3, 4, 5, 6, 7, 8, 9, 10, 14, 15, 28, 30 or more days. In some instances, the antibody and the additional anti-cancer agent are administered in 1 dose, 2 doses, 3 doses, 4 doses, 5 doses, 6 doses or more. In some instances, the antibody and the additional anti-cancer agent are administered at a therapeutically effective amount. In some instances, the cancer cell is a breast cancer cell, a kidney cancer cell, a lung cancer cell, an ovarian cancer cell, or a colorectal cancer cell. In some instances, the cancer cell is a malignant B cell.

Disclosed herein, in some embodiments, are pharmaceutical compositions comprising: (a) an antibody that selectively binds to a complex comprising a non-classical HLA-I and a neoantigen; (b) an additional anti-cancer agent; and (c) a pharmaceutically acceptable carrier or excipient. In some instances, the pharmaceutical compositions disclosed herein are for use in treating a cancer. In some instances, the pharmaceutical compositions disclosed herein are for use in treating a cancer characterized by expression of CD94/NKG2A inhibitory receptor. In some instances, the pharmaceutical compositions disclosed herein are for use in treating a cancer characterized by overexpression of CD94/NKG2A inhibitory receptor.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an exemplary schematic of a strategy to leverage the ability of an anti-HLA-E-peptide antibody to block the inhibitory signaling on immune cells and to enable cancer cell death.

FIG. 2 exemplifies anti-HLA-E-VMAPRTLFL antibody-mediated immune cell activation in peripheral blood mononuclear cells (PBMCs). Cytotoxicity assays were performed in round bottom 96-well plates, containing 1×10⁴ target cells. PBMCs from a healthy donor (Stem Cells Technology) were stained with 0.05 μM Calcein AM in RPMI for 1 min at room temperature in a volume of 10 mL. Cells were then washed twice in complete medium and used in the flow cytometry-based cytotoxicity assays. Purified antibodies and 15×10⁴ PBMCs were added to the plates for 14 hours. Additional wells were used for the assessment of spontaneous apoptosis (target cells only and maximum target cell death (target cells only in 100 μL of complete medium plus 100 μL of 100% ethanol). 10 min before acquisition, 1 μL of 5 μM SYTOX red (Thermo Fisher Scientific) was added to each tube.

FIG. 3A-FIG. 3B exemplifies combination cancer therapy mediated increase in cancer cell death. FIG. 3A exemplifies anti-HLA-E-VMAPRTLFL antibody-mediated increase in anti-CD20 and anti-PD-L1 mediated natural killer (NK) cells degranulation. JVM2 resuspended at 2.10⁴ cells/well were stimulated with the indicated antibodies for 10 minutes. 1.10⁵ primary NK cells were added to the wells and CD107a-Alexa647 antibody was added directly to the wells. Cells were incubated for 1 h, after which brefeldin A (Sigma) and Golgi-Stop (BD Biosciences) were added and the cells were incubated for an additional 5 h. Cells were stained for surface NK cell markers CD56-PE for 30 min. FIG. 3B exemplifies anti-HLA-E-VMAPRTLFL antibody-mediated increase in anti-CD20 mediated NK-92 degranulation. EB1 resuspended at 2.10⁴ cells/well were stimulated with the indicated antibodies for 10 minutes. 1.10⁵ NK-92 cells were added to the wells and CD107a-Alexa647 antibody was added directly to the wells. Cells were incubated for 1 h, after which brefeldin A (Sigma) and Golgi-Stop (BD Biosciences) were added and the cells were incubated for an additional 5 h. Cells were stained for surface NK cell markers CD56-PE for 30 min.

### DETAILED DESCRIPTION OF THE DISCLOSURE

Disclosed herein, in certain embodiments, are methods of treating cancer characterized by expression of CD94/NKG2A inhibitory receptor in an individual in need thereof, comprising administering to the individual an antibody that selectively binds to a complex comprising a non-classical HLA-I and a neoantigen. Further disclosed herein, in some embodiments, are methods of treating cancer characterized by expression of CD94/NKG2A inhibitory receptor in an individual in need thereof, comprising administering to the individual an antibody that selectively binds to a complex comprising an HLA-E and a neoantigen.

Disclosed herein, in some embodiments, are methods of treating cancer in an individual in need thereof, comprising administering to the individual: (a) an antibody that selectively binds to a complex comprising a non-classical HLA-I and a neoantigen, and (b) an additional anti-cancer agent. Further disclosed herein, in some embodiments, are methods of treating cancer in an individual in need thereof, comprising administering to the individual: (a) an antibody that selectively binds to a complex comprising a HLA-E and a neoantigen, and (b) an additional anti-cancer agent.

Disclosed herein, in some embodiments, are methods of inducing cell death of a cancer cell in a tumor microenvironment characterized by expression of CD94/NKG2A inhibitory receptor in an individual in need thereof, comprising administering to the individual an antibody that selectively binds to a complex comprising a non-classical HLA-I and a neoantigen, wherein the complex is expressed by the cancer cell. Further disclosed herein, in some embodiments, are methods of inducing cell death of a cancer cell in a tumor microenvironment characterized by expression of CD94/NKG2A inhibitory receptor in an individual in need thereof, comprising administering to the individual an antibody that selectively binds to a complex comprising an HLA-E and a neoantigen, wherein the complex is expressed by the cancer cell.

Disclosed herein, in some embodiments, are pharmaceutical compositions comprising: (a) an antibody that selectively binds to a complex comprising a non-classical HLA-I and a neoantigen; (b) an additional anti-cancer agent; and (c) a pharmaceutically acceptable carrier or excipient.

Traditional approaches to the treatment of cancers have included surgery, radiation, chemotherapy and hormone therapy. However, such therapies have not proven effective by themselves. Development of alternate remedies for preventing and/or treating cancer is crucial. More recently immunotherapy and gene therapy approaches utilizing antibodies and T-lymphocytes have emerged as new and promising methods for treating cancer.

Major histocompatibility complex (MHC) molecules, designated human leukocyte antigen (HLA) in humans, play a critical role in the body's recognition of disease and the resulting immune response to cancer and invading antigens. The HLA gene family is divided into two subgroups namely HLA Class I (HLA-I) and HLA Class II (HLA-II), with HLA-I further divided into classical HLA-I and non-classical HLA-I. Each HLA molecule forms a complex with one peptide from within the cell. On cancer cells, some of the peptide/HLA complexes are uniquely presented which enables the immune system to recognize and kill these cells. Cancer cells show a downregulation in classical HLA-I expression but an upregulation in non-classical HLA-I expression (e.g. HLA-E). Thus, the upregulated uniquely presented non-classical HLA-I-peptide complexes on cancer cells are novel targets for developing innovative immunotherapies for treatment of cancer.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. The section headings used herein are for organizational purposes only and are not to be construed as limiting the subject matter described.

As used herein, singular forms “a”, “and,” and “the” include plural referents unless the context clearly indicates otherwise. Thus, for example, reference to “an antibody” includes a plurality of antibodies and reference to “an antibody” in some embodiments includes multiple antibodies, and so forth.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

“About” a number, as used herein, refers to range including the number and ranging from 10% below that number to 10% above that number. “About” a range refers to 10% below the lower limit of the range, spanning to 10% above the upper limit of the range.

As used herein, the term “MHC” refers to the Major Histocompability Complex, which is a set of gene loci specifying major histocompatibility antigens. The term “HLA” as used herein refer to Human Leukocyte Antigens, which are the histocompatibility antigens found in humans. As used herein, “HLA” is the human form of “MHC” and the terms are used interchangeably.

As used herein “antibody” refers to a glycoprotein which exhibits binding specificity to a specific antigen. Antibodies herein also include “antigen binding portion” or fragments of the antibody that are capable of binding to the antigen. The term includes, but is not limited to, polyclonal, monoclonal, monospecific, multispecific (e.g., bispecific antibodies), natural, humanized, human, chimeric, synthetic, recombinant, hybrid, mutated, grafted, antibody fragments (e.g., a portion of a full-length antibody, generally the antigen binding or variable region thereof, e.g., Fab, Fab′, F(ab′)2, and Fv fragments), and in vitro generated antibodies so long as they exhibit the desired biological activity. The term also includes single chain antibodies, e.g., single chain Fv (sFv or scFv) antibodies, in which a variable heavy and a variable light chain are joined together (directly or through a peptide linker) to form a continuous polypeptide.

As used herein, the term “selectively binds” in the context of any binding agent, e.g., an antibody, refers to a binding agent that binds specifically to an antigen or epitope, such as with a high affinity, and does not significantly bind other unrelated antigens or epitopes.

As used herein the term “neoantigen” or “neopeptide” are used interchangeably and refer to a peptide expressed by a diseased or stressed cell (e.g. cancer cell).

As used herein, the term “immunogen” refers to a moiety, which optionally can be administered to a subject, which induces an immunological response.

The terms “recipient”, “individual”, “subject”, “host”, and “patient”, are used interchangeably herein and in some cases, refer to any mammalian subject for whom diagnosis, treatment, or therapy is desired, particularly humans. None of these terms require the supervision of medical personnel.

As used herein, the terms “treatment,” “treating,” and the like, in some cases, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a disease or disorder (e.g. cancer) in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of a cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with diseases (e.g. cancer). The term “therapeutic effect” refers to the reduction, elimination, or prevention of the disease, symptoms of the disease, or side effects of the disease in the subject.

As used herein, the term “anti-cancer agent” refers to therapeutic agents and therapies (e.g. radiation therapy) used in the treatment of cancer. Examples of therapeutic agents include, but are not limited to, monoclonal antibodies, bispecific antibodies, antibody-drug conjugates, small molecules, chimeric antigen receptor T cells (CAR-Ts), oncolytic viruses or vaccines, and chemotherapeutic agents.

### Major Histocompability Complex (MHC) or Human Leukocyte Antigens (HLA)

Major histocompatibility complexes (MHC), also termed Human Leukocyte Antigens (HLA) in humans are glycoproteins expressed on the surface of nucleated cells that act as proteomic scanning chips by providing insight into the status of cellular health. They continuously sample peptides from normal host cellular proteins, cancer cells, inflamed cells and bacterial, viral and parasite infected cells and present short peptides on the surface of cells for recognition by T lymphocytes. Presented peptides can also be derived from proteins that are out of frame or from sequences embedded in the introns, or from proteins whose translation is initiated at codons other than the conventional methionine codon, ATG.

There are two classes of MHCs in mice and humans, namely MHC I and MHC II. MHC I comprises classical and non-classical MHC I sub groups.

### Classical Major Histocompatibility Complex I (MHC I) or HLA-I

Classical MHC I molecules include HLA-A, HLA-B and HLA-C in humans and H-2-K, H-2-D, H-2-B and H-2-L in mice. Classical MHC I molecules are highly polymorphic with more than 2,735 alleles of HLA-A, 3,455 alleles of HLA-B and 2,259 alleles of HLA-C. Classical MHC I is expressed on the surface of all nucleated cells and present peptides to CD8 T lymphocytes. 30% of the proteins in the cellular machinery are rapidly degraded and are primary substrates for classical MHC I antigen presentation.

For peptide to be presented by classical MHC I molecules, proteins are first processed through the conventional processing route (ubiquitin proteasome system) that begins with the proteasome. The breakdown products (2 to 25 amino acid residues) in length are released into the cytosol. Selected cytosolic peptides are then transported into endoplasmic reticulum via Transporter associated protein (TAP) complex. TAP belongs consists of heterodimeric subunits, TAP1 and TAP2, and both bind to a transmembrane adapter chaperon glycoprotein called tapasin. Endoplasmic reticulum amino peptidase (ERAAP) in the endoplasmic reticulum trims amino-terminally extended precursors delivered by TAP to generate peptides of 8-10 amino acids in length that load onto classical MHC I molecules. Thus, the conventional processing route begins with protein degradation in the proteasome and TAP dependent transport of peptides into the endoplasmic reticulum (ER) and ends with the loading of peptides into the HLA peptide binding pocket. The proteins that contribute to the conventional processing route are collectively known as antigen processing machinery (APM) and include the proteasome, Transporter associated protein (TAP) complex, tapasin, endoplasmic reticulum amino peptidase (ERAAP), binding immunoglobulin protein (BiP), clanexin and calreticulin. Cells lacking proteasome subunits, TAP1/2, ErP57 or calreticulin have reduced numbers of classical MHC I molecules on their surface.

### Non-Classical MHC I or HLA-I

Non-classical MHC I molecules include HLA-E, HLA-F and HLA-G, and have limited polymorphisms. They play a role in regulating innate and adaptive immune responses. Non-classical MHC I molecules present peptides generated by both the conventional processing route and the alternative processing route in health and disease states, and represent a novel set of markers for targeting in disease states (e.g. cancer).

### HLA-E

The non-classical MHC class I molecule, HLA-E is non-polymorphic. In nature, 13 HLA-E alleles have been identified with only two functional variants, namely HLAE* 0101 and HLA-E*0103. The difference between HLA-E*0101 (HLA-E^{107R}) and *0103 (HLA-E^{107G}) is a single amino acid difference at position 107 which is outside the peptide binding pocket. Similar to the classical MHC I molecules, HLA-E is expressed in all cells with a nucleus, however at usually lower levels. HLA-E molecule expression in cells and tissues is generally increased during stress and disease.

In healthy cells, HLA-E presents peptides derived from classical MHC molecules to either inhibit or stimulate the activity of NK cells and a subset of CD8 T cells through engaging the receptor CD94/NKG2. The human non-classical MHC class I molecule HLA-E is a ligand for both an inhibitory NK cell receptor (CD94/NKG2A) and an activating receptor (CD94/NKG2C). Depending on the particular peptide presented by HLA-E, the HLA-E complex engages either CD94/NKG2A to inhibit NK cells and a subset of CD8 T cells or engages CD94/NKG2C to activate NK cells and a subset of CD8 T cells. Subtle changes in peptide conformation affect recognition of the HLA-E-peptide complex by the CD94/NKG2 Natural Killer cell receptors.

In healthy cells, HLA-E binds peptides that are generally 9 to 11 amino acids in length and exhibit a high degree of hydrophobicity. Unlike peptides that bind to classical MHC I molecules that usually have 2 or 3 anchor residues within the peptide sequence, non-classical HLA-E binds peptides through interaction via 5 anchor positions, namely p2, 3, 6, 7 and 9. Peptide complexes bound to HLA-E show amino acids at P5 and P8 protruding out from the binding pocket. Moreover, because more residues of the peptide are anchor peptides, the binding pocket of HLA-E with peptide binding has several deep pockets that may be targeted by small highly specific binding molecules. In contrast, the two protruding amino acids (p5 and p8) interact with CD94/NKG2 receptors on both NK cells and a subset of CD8+ T cells.

Another signal peptide that has characteristics in common with signal peptides generated from classical HLA-I molecules is the signal peptide generated from non-classical HLA-G. HLA-G expression under normal physiologic conditions is tightly regulated, with limited expression found in relatively few tissues and cells in the body. HLA-G plays a key role as an immune tolerant molecule and its expression is observed in cancer tissue/cells. Moreover, the signal peptide from HLA-G is processed by the conventional antigen processing pathway and delivered to the endoplasmic reticulum by the peptide transporter TAP. In some instances, the signal peptide is VMAPRTLFL.

HLA-E Expression and Peptide Presentation in Cancer Cells

Cells deficient in one or more components of the antigen processing machinery (APM) (e.g. proteasome, tapasin, or TAP) load peptides into MHC class I molecules via alternative processing routes which are independent of the APM-dependent conventional processing route. APM-deficient cells not only have reduced numbers of classical MHC I molecules on their surface, but also show an increase in the cell surface density of HLA-E molecules as well as an increase in the repertoire of peptides presented. The alternative processing routes are constitutively turned on and produce peptides in both healthy and diseased cells. These peptides, however, are not presented by healthy cells; instead they are only presented in diseased or stressed cells. As such, the different peptide repertoires generated by APM-defective cells, also known as “T-cell epitopes associated with impaired peptide processing” (TEIPP), represent novel targets unique to cancer cells, and represent ideal targets for therapeutic development in the treatment of cancer.

### Classical Major Histocompatibility Complex II (MHC II) or HLA-II

MHC II molecules in humans include HLA-DM, HLA-DO, HLA-DP, HLA-DQ and HLA-DR and include H-2 I-A and H-2 I-E in mice. MHC II expression is more restricted to B cells, dendritic cells, macrophages, activated T cells and thymic epithelial cells and MHC II molecules present peptides to CD4 lymphocytes.

### Antibodies to Target Non-Classical HLA-I/Cancer Peptides

Disclosed herein, in certain embodiments, are compositions that target a complex comprising a non-classical HLA-I and a neoantigen, and methods of use thereof. In some instances, the compositions comprise antibodies. In some instances, the antibodies are scFvs from mice and human libraries. In some instances, the antibodies are single domain antibodies derived from immunized llamas.

Disclosed herein, in certain embodiments, are antibodies that selectively bind to a complex comprising a non-classical HLA-I and a peptide. In some instances, the antibody does not have a binding affinity to the non-classical HLA-I alone. In some instances, the antibody does not have a binding affinity to the peptide alone. In some instances, the antibody does not have a binding affinity to a complex comprising the non-classical HLA-I and a non-relevant peptide.

In some instances, the peptide comprises, consists essentially of, or consists of a sequence VMAPRTLFL. In some instances, the non-classical HLA-I is HLA-E, HLA-F, HLA-G, or HLA-H. In some instances, the non-classical HLA-I is HLA-E. In some instances, the HLA-E is HLA-E*0101. In some instances, the HLA-E is HLA-E*0103. In some instances, the antibody selectively binds to the complex comprising the HLA-E and the peptide. In some instances, the antibody selectively binds to the complex comprising the HLA-E*0101 and the peptide. In some instances, the antibody selectively binds to the complex comprising the HLA-E*0103 and the peptide. In some instances, the antibody selectively binds to the complex comprising the HLA-E*0101 and the peptide, and to the complex comprising the HLA-E*0103 and the peptide. In some instances, the complex comprises the HLA-E and VMAPRTLFL.

In some instances, the antibody is a murine antibody. In some instances, the antibody is a chimeric antibody. In some instances, the antibody is a camelid antibody. In some instances, the antibody is a humanized antibody. In some instances, the antibody is a human antibody. In some instances, the antibody is a TCR-like antibody. In some instances, the antibody is a single domain antibody. In some instances, the single domain antibody is a camelid single domain antibody. In some instances, the antibody is a multispecific antibody. In some instances, the antibody is a multifunctional antibody.

In some instances, the selective binding of the antibody to the complex comprising the non-classical HLA-I and the neoantigen inhibits the binding of the complex to the CD94/NKG2A inhibitory receptor. In some instances, the inhibition in binding of the complex to the CD94/NKG2A inhibitory receptor induces activation of natural killer (NK) cells. In some instances, the inhibition in binding of the complex to the CD94/NKG2A inhibitory receptor induces activation of CD8+ T cells. In some instances, the inhibition in binding of the complex to the CD94/NKG2A inhibitory receptor induces cell death of a cell expressing the non-classical HLA-I and the neoantigen. In some instances, the cell death is apoptotic cell death. In some instances, the cell death is non-apoptotic cell death. In some instances, the cell is a cancer cell.

In some instances, the cancer cell is a breast cancer cell. In some instances, the cancer cell is a kidney cancer cell. In some instances, the cancer cell is a lung cancer cell. In some instances, the cancer cell is an ovarian cancer cell. In some instances, the cancer cell is a colorectal cancer cell. In some instances, the cancer cell is a malignant B cell.

### Methods of Treatment

Disclosed herein, in some embodiments, are methods of treating cancer characterized by expression of CD94/NKG2A inhibitory receptor in an individual in need thereof, comprising administering to the individual an antibody that selectively binds to a complex comprising a non-classical HLA-I and a neoantigen. Further disclosed herein, in some embodiments, are methods of treating cancer characterized by expression of CD94/NKG2A inhibitory receptor in an individual in need thereof, comprising administering to the individual an antibody that selectively binds to a complex comprising an HLA-E and a neoantigen.

Also disclosed herein, in some embodiments, are methods of inducing cell death of a cancer cell in a tumor microenvironment characterized by expression of CD94/NKG2A inhibitory receptor in an individual in need thereof, comprising administering to the individual an antibody that selectively binds to a complex comprising a non-classical HLA-I and a neoantigen, wherein the complex is expressed by the cancer cell. Further disclosed herein, in some embodiments, are methods of inducing cell death of a cancer cell in a tumor microenvironment characterized by expression of CD94/NKG2A inhibitory receptor in an individual in need thereof, comprising administering to the individual an antibody that selectively binds to a complex comprising an HLA-E and a neoantigen, wherein the complex is expressed by the cancer cell.

In some instances, the cancer is characterized by the overexpression of the CD94/NKG2A inhibitory receptor. In some instances, the tumor microenvironment is characterized by overexpression of the CD94/NKG2A inhibitory receptor. In some instances, the methods further comprise assaying for expression or overexpression of the CD94/NKG2A inhibitory receptor in the individual.

In some instances, the antibody does not have a binding affinity to (i) the non-classical HLA-I alone; or (ii) the neoantigen alone. In some instances, the neoantigen comprises, consists essentially of, or consists of a sequence VMAPRTLFL. In some instances, the non-classical HLA-I is HLA-E, HLA-F, HLA-G, or HLA-H. In some instances, the non-classical HLA-I is HLA-E. In some instances, the HLA-E is HLA-E*0101 or HLA-E*0103. In some instances, the antibody selectively binds to the complex comprising the HLA-E and the neoantigen. In some instances, the antibody selectively binds to the complex comprising: (a) the HLA-E*0101 and the neoantigen; (b) the HLA-E*0103 and the neoantigen; or (c) the HLA-E*0101 and the neoantigen, and the HLA-E*0103 and the neoantigen. In some instances, the antibody does not have a binding affinity to (i) the HLA-E alone; or (ii) the neoantigen alone. In some instances, the complex comprises the HLA-E and VMAPRTLFL.

In some instances, the antibody is a murine antibody. In some instances, the antibody is a chimeric antibody. In some instances, the antibody is a camelid antibody. In some instances, the antibody is a humanized antibody. In some instances, the antibody is a human antibody. In some instances, the antibody is a TCR-like antibody. In some instances, the antibody is a single domain antibody. In some instances, the single domain antibody is a camelid single domain antibody. In some instances, the antibody is a multispecific antibody. In some instances, the antibody is a multifunctional antibody.

In some instances, the selective binding of the antibody to the complex comprising the non-classical HLA-I and the neoantigen inhibits the binding of the complex to the CD94/NKG2A inhibitory receptor. In some instances, the selective binding of the antibody to the complex comprising the HLA-E and the neoantigen inhibits the binding of the complex to the CD94/NKG2A inhibitory receptor. In some instances, the inhibition in binding of the complex to the CD94/NKG2A inhibitory receptor induces activation of natural killer (NK) cells. In some instances, the inhibition in binding of the complex to the CD94/NKG2A inhibitory receptor induces activation of CD8+ T cells. In some instances, the inhibition in binding of the complex to the CD94/NKG2A inhibitory receptor induces cell death of a cell expressing the non-classical HLA-I and the neoantigen. In some instances, the inhibition in binding of the complex to the CD94/NKG2A inhibitory receptor induces cell death of a cell expressing the HLA-E and the neoantigen. In some instances, the cell death is apoptotic cell death. In some instances, the cell death is non-apoptotic cell death. In some instances, the cell is a cancer cell.

In some instances, the methods further comprise administering an additional anti-cancer agent. In some instances, the antibody and the additional anti-cancer agent have a synergistic effect. In some instances, the additional anti-cancer agent comprises CD20 inhibitor, HER-2 inhibitor, CD52 inhibitor, EGFR inhibitor, VEGF inhibitor, CCR4 inhibitor, PD-L1 inhibitor, SLAMF7 inhibitor, CD38 inhibitor, GD2 inhibitor, PTK-7 inhibitor, P-cadherin inhibitor, MCSF inhibitor, IDO1 inhibitor, CCR2 inhibitor, CXCR4 inhibitor, PD-1 inhibitor, CTLA-4 inhibitor, OX40 agonist, 4-1BB agonist, androgen receptor inhibitor, tyrosine kinase inhibitor, PARP inhibitor, chimeric antigen receptor T cells (CAR-T cells), oncolytic virus, and any combination thereof. In some instances, the additional anti-cancer agent is CD20 inhibitor. In some instances, the additional anti-cancer agent is PD-L1 inhibitor. In some instances, the additional anti-cancer agent comprises Rituximab, Trastuzumab, Alemutuzumab, Cetixumab, Bevacizumab, Panitumumab, Obinutuzumab, Mogamulizumab, Necitumumab, Atezolizumab, Elotuzumab, Daratumumab, Dinutuximab, and any combination thereof. In some embodiments, the anti-cancer agent comprises a chemotherapeutic agent. In some embodiments, the chemotherapeutic agents include, among others, cytotoxic agents, anti-metabolite agents (e.g., folate antagonists, purine analogs, pyrimidine analogs, etc.), topoisomerase inhibitors (e.g., camptothecin derivatives, anthracenedione, anthracyclines, epipodophyllotoxins, quinoline alkaloids, etc.), anti-microtubule agents (e.g., taxanes, vinca alkaloids), protein synthesis inhibitors (e.g., cephalotaxine, camptothecin derivatives, quinoline alkaloids), alkylating agents (e.g., alkyl sulfonates, ethylenimines, nitrogen mustards, nitrosoureas, platinum derivatives, triazenes, etc.), alkaloids, terpenoids, and kinase inhibitors.

In some instances, the antibody and the additional anti-cancer agent are administered concurrently. In some instances, the antibody and the additional anti-cancer agent are administered sequentially. In some instances, the antibody is administered prior to the additional anti-cancer agent. In some instances, the antibody is administered after the additional anti-cancer agent. In some instances, the antibody and the additional anti-cancer agent are administered in a unified dosage form. In some instances, the antibody and the additional anti-cancer agent are administered in a separate dosage form.

In some instances, the antibody is administered continuously for 1, 2, 3, 4, 5, 6, 7, 8, 9, 10, 14, 15, 28, 30 or more days. In some instances, the antibody is administered at predetermined time intervals for 1, 2, 3, 4, 5, 6, 7, 8, 9, 10, 14, 15, 28, 30 or more days. In some instances, the antibody is administered intermittently for 1, 2, 3, 4, 5, 6, 7, 8, 9, 10, 14, 15, 28, 30 or more days. In some instances, the antibody is administered in 1 dose, 2 doses, 3 doses, 4 doses, 5 doses, 6 doses or more. In some instances, the antibody is administered at a therapeutically effective amount.

In some instances, the additional anti-cancer agent is administered continuously for 1, 2, 3, 4, 5, 6, 7, 8, 9, 10, 14, 15, 28, 30 or more days. In some instances, the additional anti-cancer agent is administered at predetermined time intervals for 1, 2, 3, 4, 5, 6, 7, 8, 9, 10, 14, 15, 28, 30 or more days. In some instances, the additional anti-cancer agent is administered intermittently for 1, 2, 3, 4, 5, 6, 7, 8, 9, 10, 14, 15, 28, 30 or more days. In some instances, the additional anti-cancer agent is administered in 1 dose, 2 doses, 3 doses, 4 doses, 5 doses, 6 doses or more. In some instances, the additional anti-cancer agent is administered at a therapeutically effective amount.

In some instances, the cancer is breast cancer. In some instances, the cancer is kidney cancer. In some instances, the cancer is lung cancer. In some instances, the cancer is ovarian cancer. In some instances, the cancer is colorectal cancer. In some instances, the cancer is a B-cell malignancy. In some instances, the cancer cell is a breast cancer cell. In some instances, the cancer cell is a kidney cancer cell. In some instances, the cancer cell is a lung cancer cell. In some instances, the cancer cell is an ovarian cancer cell. In some instances, the cancer cell is a colorectal cancer cell. In some instances, the cancer cell is a malignant B cell.

### Combination Cancer Therapy

Disclosed herein, in some embodiments, are methods of treating cancer in an individual in need thereof, comprising administering to the individual: (a) an antibody that selectively binds to a complex comprising a non-classical HLA-I and a neoantigen, and (b) an additional anti-cancer agent. Further disclosed herein, in some embodiments, are methods of treating cancer in an individual in need thereof, comprising administering to the individual: (a) an antibody that selectively binds to a complex comprising a HLA-E and a neoantigen, and (b) an additional anti-cancer agent.

In some instances, the antibody does not have a binding affinity to (i) the non-classical HLA-I alone; or (ii) the neoantigen alone. In some instances, the neoantigen comprises, consists essentially of, or consists of a sequence VMAPRTLFL. In some instances, the non-classical HLA-I is HLA-E, HLA-F, HLA-G, or HLA-H. In some instances, the non-classical HLA-I is HLA-E. In some instances, the HLA-E is HLA-E*0101 or HLA-E*0103. In some instances, the antibody selectively binds to the complex comprising the HLA-E and the neoantigen. In some instances, the antibody selectively binds to the complex comprising: (a) the HLA-E*0101 and the neoantigen; (b) the HLA-E*0103 and the neoantigen; or (c) the HLA-E*0101 and the neoantigen, and the HLA-E*0103 and the neoantigen. In some instances, the antibody does not have a binding affinity to (i) the HLA-E alone; or (ii) the neoantigen alone. In some instances, the complex comprises the HLA-E and VMAPRTLFL.

In some instances, the cancer is characterized by expression of CD94/NKG2A inhibitory receptor. In some instances, the cancer is characterized by overexpression of the CD94/NKG2A inhibitory receptor. In some instances, the methods further comprise assaying for expression or overexpression of the CD94/NKG2A inhibitory receptor in the individual.

In some instances, the selective binding of the antibody to the complex comprising the non-classical HLA-I and the neoantigen inhibits the binding of the complex to the CD94/NKG2A inhibitory receptor. In some instances, the selective binding of the antibody to the complex comprising the HLA-E and the neoantigen inhibits the binding of the complex to the CD94/NKG2A inhibitory receptor. In some instances, the inhibition in binding of the complex to the CD94/NKG2A inhibitory receptor induces activation of natural killer (NK) cells. In some instances, the inhibition in binding of the complex to the CD94/NKG2A inhibitory receptor induces activation of CD8+ T cells. In some instances, the inhibition in binding of the complex to the CD94/NKG2A inhibitory receptor induces cell death of a cell expressing the non-classical HLA-I and the neoantigen. In some instances, the inhibition in binding of the complex to the CD94/NKG2A inhibitory receptor induces cell death of a cell expressing the HLA-E and the neoantigen. In some instances, the cell death is apoptotic cell death. In some instances, the cell death is non-apoptotic cell death. In some instances, the cell is a cancer cell.

In some instances, the antibody is a murine antibody. In some instances, the antibody is a chimeric antibody. In some instances, the antibody is a camelid antibody. In some instances, the antibody is a humanized antibody. In some instances, the antibody is a human antibody. In some instances, the antibody is a TCR-like antibody. In some instances, the antibody is a single domain antibody. In some instances, the single domain antibody is a camelid single domain antibody. In some instances, the antibody is a multispecific antibody. In some instances, the antibody is a multifunctional antibody.

In some instances, the antibody and the additional anti-cancer agent have a synergistic effect. In some instances, the additional anti-cancer agent comprises CD20 inhibitor, HER-2 inhibitor, CD52 inhibitor, EGFR inhibitor, VEGF inhibitor, CCR4 inhibitor, PD-L1 inhibitor, SLAMF7 inhibitor, CD38 inhibitor, GD2 inhibitor, PTK-7 inhibitor, P-cadherin inhibitor, MCSF inhibitor, IDO1 inhibitor, CCR2 inhibitor, CXCR4 inhibitor, PD-1 inhibitor, CTLA-4 inhibitor, OX40 agonist, 4-1BB agonist, androgen receptor inhibitor, tyrosine kinase inhibitor, PARP inhibitor, chimeric antigen receptor T cells (CAR-T cells), oncolytic virus, and any combination thereof. In some instances, the additional anti-cancer agent is CD20 inhibitor. In some instances, the additional anti-cancer agent is PD-L1 inhibitor. In some instances, the additional anti-cancer agent comprises Rituximab, Trastuzumab, Alemutuzumab, Cetixumab, Bevacizumab, Panitumumab, Obinutuzumab, Mogamulizumab, Necitumumab, Atezolizumab, Elotuzumab, Daratumumab, Dinutuximab, and any combination thereof. In some embodiments, the anti-cancer agent comprises a chemotherapeutic agent. In some embodiments, the chemotherapeutic agents include, among others, cytotoxic agents, anti-metabolite agents (e.g., folate antagonists, purine analogs, pyrimidine analogs, etc.), topoisomerase inhibitors (e.g., camptothecin derivatives, anthracenedione, anthracyclines, epipodophyllotoxins, quinoline alkaloids, etc.), anti-microtubule agents (e.g., taxanes, vinca alkaloids), protein synthesis inhibitors (e.g., cephalotaxine, camptothecin derivatives, quinoline alkaloids), alkylating agents (e.g., alkyl sulfonates, ethylenimines, nitrogen mustards, nitrosoureas, platinum derivatives, triazenes, etc.), alkaloids, terpenoids, and kinase inhibitors.

In some instances, the antibody and the additional anti-cancer agent are administered concurrently. In some instances, the antibody and the additional anti-cancer agent are administered sequentially. In some instances, the antibody is administered prior to the additional anti-cancer agent. In some instances, the antibody is administered after the additional anti-cancer agent. In some instances, the antibody and the additional anti-cancer agent are administered in a unified dosage form. In some instances, the antibody and the additional anti-cancer agent are administered in a separate dosage form.

In some instances, the antibody is administered continuously for 1, 2, 3, 4, 5, 6, 7, 8, 9, 10, 14, 15, 28, 30 or more days. In some instances, the antibody is administered at predetermined time intervals for 1, 2, 3, 4, 5, 6, 7, 8, 9, 10, 14, 15, 28, 30 or more days. In some instances, the antibody is administered intermittently for 1, 2, 3, 4, 5, 6, 7, 8, 9, 10, 14, 15, 28, 30 or more days. In some instances, the antibody is administered in 1 dose, 2 doses, 3 doses, 4 doses, 5 doses, 6 doses or more. In some instances, the antibody is administered at a therapeutically effective amount.

In some instances, the additional anti-cancer agent is administered continuously for 1, 2, 3, 4, 5, 6, 7, 8, 9, 10, 14, 15, 28, 30 or more days. In some instances, the additional anti-cancer agent is administered at predetermined time intervals for 1, 2, 3, 4, 5, 6, 7, 8, 9, 10, 14, 15, 28, 30 or more days. In some instances, the additional anti-cancer agent is administered intermittently for 1, 2, 3, 4, 5, 6, 7, 8, 9, 10, 14, 15, 28, 30 or more days. In some instances, the additional anti-cancer agent is administered in 1 dose, 2 doses, 3 doses, 4 doses, 5 doses, 6 doses or more. In some instances, the additional anti-cancer agent is administered at a therapeutically effective amount.

In some instances, the cancer is breast cancer. In some instances, the cancer is kidney cancer. In some instances, the cancer is lung cancer. In some instances, the cancer is ovarian cancer. In some instances, the cancer is colorectal cancer. In some instances, the cancer is a B-cell malignancy.

### Anti-Cancer Agents

Disclosed herein, in some embodiments, are methods and compositions for treating cancer in an individual in need thereof comprising antibodies disclosed herein in combination with an additional anti-cancer agent. Further disclosed herein, in some embodiments, are methods and compositions for inducing cell death of a cancer cell in an individual in need thereof comprising antibodies disclosed herein in combination with an additional anti-cancer agent.

In some embodiments, the additional anti-cancer agent comprises a chemotherapeutic agent, a cytotoxin, a steroid, an immunotherapeutic agent, an immunomodulatory agent, an immunosuppressive agent, a targeted therapy agent, an anti-inflammatory agent, a cytokine therapy, an interferon therapy (e.g., INF-α), an interlukin therapy (e.g., IL-2, IL-7, or IL-11), a colony-stimulating factor therapy (e.g., G-CSF), an antibody therapy, a hormonal therapy, a viral therapy, gene therapy, cancer vaccines (e.g., tumor cell vaccines, antigen vaccines, dendritic cell vaccines, DNA vaccines, or vector based vaccines), an antibiotic, an antitumour antibiotic, or any combination thereof. In some embodiments, the anti-cancer agent comprises an anti-TNF agent, an IL-1 receptor antagonist, an IL-2 receptor antagonist, a T-cell co-stimulatory blocker, a B cell depleting agent, an alkylating agent, an anti-metabolite, a plant alkaloid, a terpenoids, a topoisomerase inhibitor, an anti-diabetes agent, a leukotriene inhibitor, or combinations thereof. In some embodiments, the additional anti-cancer agent comprises a B cell receptor pathway inhibitor, a CD79A inhibitor, a CD79B inhibitor, a CD19 inhibitor, a Lyn inhibitor, a Syk inhibitor, a PI3K inhibitor, a Blnk inhibitor, a PLCγ inhibitor, a PKCβ inhibitor, an IAP inhibitor, an mTOR inhibitor, a radioimmunotherapeutic, a DNA damaging agent, a proteosome inhibitor, a histone deacytlase inhibitor, a protein kinase inhibitor, a hedgehog inhibitor, an Hsp90 inhibitor, a telomerase inhibitor, a Jak1/2 inhibitor, a protease inhibitor, a PKC inhibitor, a PARP inhibitor, or a combination thereof.

In some embodiments, the additional anti-cancer agent comprises anti-hormonal agents that act to regulate or inhibit hormone action on tumors such as anti-estrogens including for example tamoxifen, raloxifene; aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Further anti-cancer agents include sorafenib and other protein kinase inhibitors such as afatinib, axitinib, bevacizumab, cetuximab, crizotinib, dasatinib, erlotinib, fostamatinib, gefitinib, imatinib, lapatinib, lenvatinib, mubritinib, nilotinib, panitumumab, pazopanib, pegaptanib, ranibizumab, ruxolitinib, trastuzumab, vandetanib, vemurafenib, and sunitinib; sirolimus (rapamycin), everolimus and other mTOR inhibitors.

Examples of additional anti-cancer agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea). Moreover, exemplary anti-cancer agents that disrupt cell replication include: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib); NF-κB inhibitors, including inhibitors of IκB kinase; antibodies which bind to proteins overexpressed in cancers and other inhibitors of proteins or enzymes known to be upregulated, over-expressed or activated in cancers, the inhibition of which downregulates cell replication.

Examples of additional anti-cancer agents further include alkylating agents such as thiotepa and cyclophosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL™, Bristol-Myers Squibb Oncology, Princeton, N.J.) and doxetaxel (TAXOTERE™, Rhne-Poulenc Rorer, Antony, France); chlorambucil; gemcitabine; 6-thioguanine; mercaptopurine; methotrexate; platinum analogs such as cisplatin and carboplatin; vinblastine; trastuzumab, docetaxel, platinum; etoposide (VP-16); Ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAKT™ (denileukin diftitox); esperamicins; capecitabine; and pharmaceutically acceptable salts, acids or derivatives of any of the above.

In some embodiments, the additional anti-cancer agent comprises alefacept, efalizumab, methotrexate, acitretin, isotretinoin, hydroxyurea, mycophenolate mofetil, sulfasalazine, 6-Thioguanine, Dovonex, Taclonex, betamethasone, tazarotene, hydroxychloroquine, etanercept, adalimumab, infliximab, abatacept, rituximab, tratuzumab, Anti-CD45 monoclonal antibody AHN-12 (NCI), Iodine-131 Anti-B1 Antibody (Corixa Corp.), anti-CD66 monoclonal antibody BW 250/183 (NCI, Southampton General Hospital), anti-CD45 monoclonal antibody (NCI, Baylor College of Medicine), antibody anti-anb3 integrin (NCI), BIW-8962 (BioWa Inc.), Antibody BC8 (NCI), antibody muJ591 (NCI), indium In 111 monoclonal antibody MN-14 (NCI), yttrium Y 90 monoclonal antibody MN-14 (NCI), F105 Monoclonal Antibody (MAID), Monoclonal Antibody RAV12 (Raven Biotechnologies), CAT-192 (Human Anti-TGF-Beta1 Monoclonal Antibody, Genzyme), antibody 3F8 (NCI), 177Lu-J591 (Weill Medical College of Cornell University), TB-403 (BioInvent International AB), anakinra, azathioprine, cyclophosphamide, cyclosporine A, leflunomide, d-penicillamine, amitriptyline, or nortriptyline, chlorambucil, nitrogen mustard, prasterone, LJP 394 (abetimus sodium), LJP 1082 (La Jolla Pharmaceutical), eculizumab, belibumab, rhuCD40L (MAID), epratuzumab, sirolimus, tacrolimus, pimecrolimus, thalidomide, antithymocyte globulin-equine (Atgam, Pharmacia Upjohn), antithymocyte globulin-rabbit (Thymoglobulin, Genzyme), Muromonab-CD3 (FDA Office of Orphan Products Development), basiliximab, daclizumab, riluzole, cladribine, natalizumab, interferon beta-lb, interferon beta-1a, tizanidine, baclofen, mesalazine, asacol, pentasa, mesalamine, balsalazide, olsalazine, 6-mercaptopurine, AIN457 (Anti IL-17 Monoclonal Antibody, Novartis), theophylline, D2E7 (a human anti-TNF mAb from Knoll Pharmaceuticals), Mepolizumab (Anti-IL-5 antibody, SB 240563), Canakinumab (Anti-IL-1 Beta Antibody, NIAMS), Anti-IL-2 Receptor Antibody (Daclizumab, NHLBI), CNTO 328 (Anti IL-6 Monoclonal Antibody, Centocor), ACZ885 (fully human anti-interleukin-lbeta monoclonal antibody, Novartis), CNTO 1275 (Fully Human Anti-IL-12 Monoclonal Antibody, Centocor), (3 S)—N-hydroxy-4-({4-[(4-hydroxy-2-butynyl)oxy]phenyl}sulfonyl)-2,2-dimet-hyl-3-thiomorpholine carboxamide (apratastat), golimumab (CNTO 148), Onercept, BG9924 (Biogen Idec), Certolizumab Pegol (CDP870, UCB Pharma), AZD9056 (AstraZeneca), AZD5069 (AstraZeneca), AZD9668 (AstraZeneca), AZD7928 (AstraZeneca), AZD2914 (AstraZeneca), AZD6067 (AstraZeneca), AZD3342 (AstraZeneca), AZD8309 (AstraZeneca), [(1R)-3-methyl-1-({(2S)-3-phenyl-2-[(pyrazin-2-ylcarbonyl)amino]propanoyl}amino)butyl]boronic acid (Bortezomib), AMG-714, (Anti-IL 15 Human Monoclonal Antibody, Amgen), ABT-874 (Anti IL-12 monoclonal antibody, Abbott Labs), MRA(Tocilizumab, an Anti IL-6 Receptor Monoclonal Antibody, Chugai Pharmaceutical), CAT-354 (a human anti-interleukin-13 monoclonal antibody, Cambridge Antibody Technology, MedImmune), aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, flurobiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, celecoxib, rofecoxib, valdecoxib, parecoxib, etoricoxib, lumiracoxib, CS-502 (Sankyo), JTE-522 (Japan Tobacco Inc.), L-745,337 (Almirall), NS398 (Sigma), betamethasone (Celestone), prednisone (Deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluprednidene, fluticasone, formocortal, formoterol, halcinonide, halometasone, hydrocortisone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, rimexolone, tixocortol, triamcinolone, ulobetasol, Pioglitazone, Rosiglitazone, Glimepiride, Glyburide, Chlorpropamide, Glipizide, Tolbutamide, Tolazamide, Glucophage, Metformin, (glyburide+metformin), Rosiglitazone+metformin, (Rosiglitazone+glimepiride), Exenatide, Insulin, Sitagliptin, (glipizide and metformin), Repaglinide, Acarbose, Nateglinide, Orlistat, cisplatin; carboplatin; oxaliplatin; mechlorethamine; cyclophosphamide; chlorambucil; vincristine; vinblastine; vinorelbine; vindesine; mercaptopurine; fludarabine; pentostatin; cladribine; 5-fluorouracil (5FU); floxuridine (FUDR); cytosine arabinoside; trimethoprim; pyrimethamine; pemetrexed; paclitaxel; docetaxel; etoposide; teniposide; irinotecan; topotecan; amsacrine; etoposide; etoposide phosphate; teniposide; dactinomycin; doxorubicin; daunorubicin; valrubicine; idarubicine; epirubicin; bleomycin; plicamycin; mitomycin; finasteride; goserelin; aminoglutethimide; anastrozole; letrozole; vorozole; exemestane; 4-androstene-3,6,17-trione (“6-OXO”; 1,4,6-androstatrien-3,17-dione (ATD); formestane; testolactone; fadrozole; A-81834 (3-(3-(1,1-dimethylethylthio-5-(quinoline-2-ylmethoxy)-1-(4-chloromethylphenyl)indole-2-yl)-2,2-dimethylpropionaldehyde oxime-O-2-acetic acid; AME103 (Amira); AME803 (Amira); atreleuton; BAY-x-1005 ((R)-(+)-alpha-cyclopentyl-4-(2-quinolinylmethoxy)-Benzeneacetic acid); CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dimethylethyl)thio]-α,α-dimethyl-5-(1-methylethyl)-1H-indole-2-propanoic acid, sodium salt); MK591 (3-(1-4[(4-chlorophenyl)methyl]-3-[(t-butylthio)-5-((2-quinoly)methoxy)-1H-indole-2]-, dimehtylpropanoic acid); RP64966 ([4-[5-(3-Phenyl-propyl)thiophen-2-yl]butoxy] acetic acid); SA6541 ((R)—S-[[4-(dimethylamino)phenyl]methyl]-N-(3-mercapto-2methyl-1-oxopropyl-L-cycteine); SC-56938 (ethyl-1-[2-[4-(phenylmethyl)phenoxy] ethyl]-4-piperidine-carboxylate); VIA-2291 (Via Pharmaceuticals); WY-47,288 (2-[(1-naphthalenyloxy)methyl]quinoline); zileuton; ZD-2138 (6-((3-fluoro-5-(tetrahydro-4-methoxy-2H-pyran-4yl)phenoxy)methyl)-1-methyl-2(1H)-quinlolinone); doxycycline; or combinations thereof.

### Pharmaceutical Compositions and Formulations

Also disclosed herein are pharmaceutical compositions comprising: (a) antibodies that selectively bind to a complex comprising a non-classical HLA-I and a neoantigen; (b) an additional anti-cancer agent; and (c) a pharmaceutically acceptable carrier or excipient. In some instances, the pharmaceutical compositions disclosed herein are for use in treating a cancer. In some instances, the pharmaceutical compositions disclosed herein are for use in treating a cancer characterized by expression of CD94/NKG2A inhibitory receptor. In some instances, the pharmaceutical compositions disclosed herein are for use in treating a cancer characterized by overexpression of CD94/NKG2A inhibitory receptor.

In some embodiments, excipients for use with the compositions disclosed herein include maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate.

Pharmaceutical formulations, in some embodiments, are made to be compatible with a particular local, regional or systemic administration or delivery route. Thus, pharmaceutical formulations include carriers, diluents, or excipients suitable for administration by particular routes. Specific non-limiting examples of routes of administration for compositions herein are parenteral, e.g., intravenous, intra-arterial, intradermal, intramuscular, subcutaneous, intra-pleural, transdermal (topical), transmucosal, intra-cranial, intra-spinal, intra-ocular, rectal, oral (alimentary), mucosal administration, and any other formulation suitable for the treatment method or administration protocol.

In some embodiments, solutions or suspensions used for parenteral application include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose. In some embodiments, pH is adjusted with acids or bases, such as hydrochloric acid or sodium hydroxide.

Pharmaceutical formulations for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In some embodiments, the carrier is a solvent or dispersion medium containing, for example, water, ethanol, polyol (for example, glycerol, propylene glycol, and liquid polyetheylene glycol, and the like), or suitable mixtures thereof. Fluidity is maintained, in some embodiments, for example, by the use of a coating such as lecithin, by the maintenance of the required particle size in the case of dispersion, and by the use of surfactants. Antibacterial and antifungal agents include, for example, parabens, chlorobutanol, phenol, ascorbic acid, and thimerosal. Isotonic agents, for example, sugars; polyalcohols such as mannitol or sorbitol; or sodium chloride, in some embodiments, are included in the composition. In some cases, also included is an agent which delays absorption, in some embodiments, for example, aluminum monostearate or gelatin prolongs absorption of injectable compositions.

In some embodiments, sterile injectable formulations are prepared by incorporating the active composition in the required amount in an appropriate solvent with one or a combination of above ingredients. Generally, dispersions are prepared by incorporating the active composition into a sterile vehicle containing a basic dispersion medium and any other ingredient. In the case of sterile powders for the preparation of sterile injectable solutions, methods of preparation include, for example, vacuum drying and freeze-drying which yields a powder of the active ingredient plus any additional desired ingredient from a previously prepared solution thereof.

For transmucosal or transdermal administration, penetrants appropriate to the barrier to be permeated are used in the formulation. Such penetrants are known in the art, and include, for example, for transmucosal administration, detergents, bile salts, and fusidic acid derivatives. In some embodiments, transmucosal administration is accomplished through the use of nasal sprays, inhalation devices (e.g., aspirators) or suppositories. For transdermal administration, the active compounds are formulated into ointments, salves, gels, creams or patches.

In some embodiments, the pharmaceutical formulations are prepared with carriers that protect against rapid elimination from the body, such as a controlled release formulation or a time delay material such as glyceryl monostearate or glyceryl stearate. The formulations, in some embodiments, are also delivered using articles of manufacture such as implants and microencapsulated delivery systems to achieve local, regional or systemic delivery or controlled or sustained release.

### Therapeutic Regimens for a Pharmaceutical Composition

In some embodiments, a pharmaceutical compositions described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, in some embodiments, the dosage or the frequency of administration, or both, is reduced, as a function of the symptoms, to a level at which the improved disease, disorder or condition is retained.

In some embodiments, the amount of a given agent that correspond to such an amount varies depending upon factors such as the particular composition, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the composition used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compositions exhibiting high therapeutic indices are preferred. The data obtained from cell culture assays and animal studies are used in formulating a range of dosage for use in human. The dosage of such composition lies preferably within a range of circulating concentrations that include the ED50 with minimal toxicity. The dosage varies within this range depending upon the dosage form employed and the route of administration utilized.

### Examples

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1. Anti-HLA-E-Peptide Antibody Mediates Activation of Immune Cells to Induce Cell Death

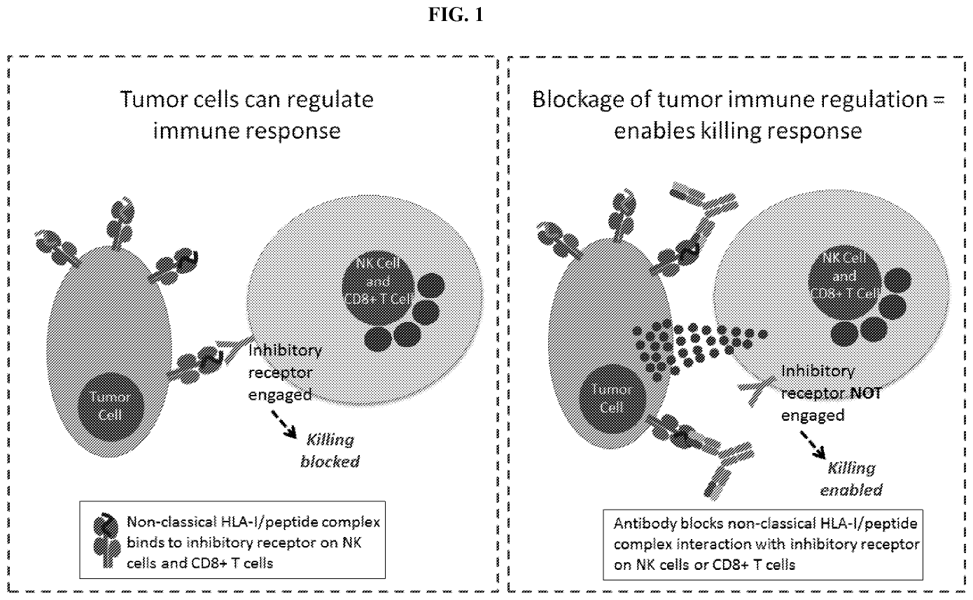

FIG. 1 is an exemplary schematic of a strategy to leverage the ability of an anti-HLA-E-peptide antibody to block the inhibitory signaling on immune cells and to enable cancer cell death.

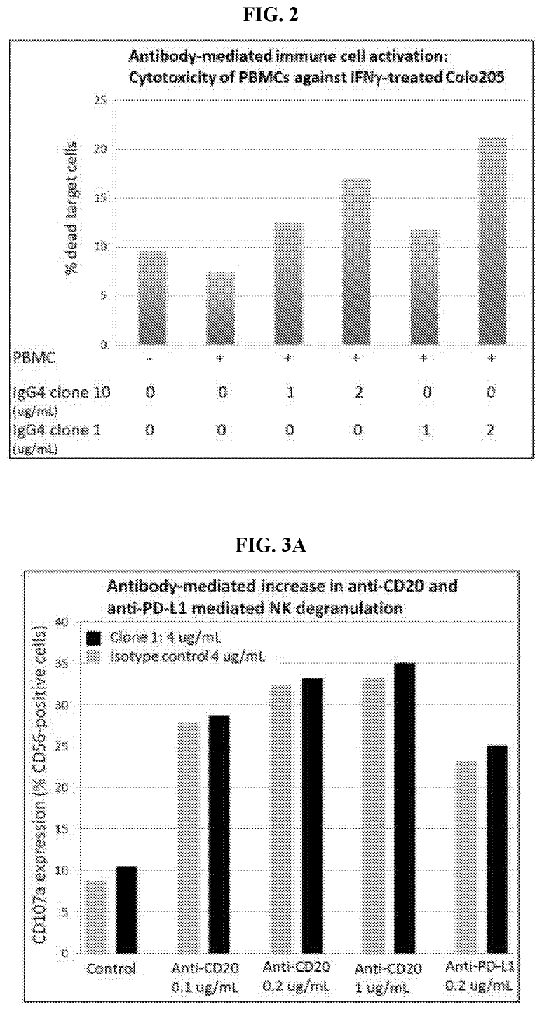

Cytotoxicity assays were performed in round bottom 96-well plates, containing 1×10⁴ target cells. PBMCs from a healthy donor (Stem Cells Technology) were stained with 0.05 μM Calcein AM in RPMI for 1 min at room temperature in a volume of 10 mL. Cells were then washed twice in complete medium and used in the flow cytometry-based cytotoxicity assays. Purified antibodies and 15×10⁴ PBMCs were added to the plates for 14 hours. Additional wells were used for the assessment of spontaneous apoptosis (target cells only and maximum target cell death (target cells only in 100 μL of complete medium plus 100 μL of 100% ethanol). 10 min before acquisition, 1 μL of 5 μM SYTOX red (Thermo Fisher Scientific) was added to each tube. FIG. 2 illustrates an increase in dead target cells in the presence of anti-HLA-E-VMAPRTLFL antibody clones.

### Example 2. Increase in Cancer Cell Death when Anti-HLA-E-Peptide Antibody is Used in Combination with an Additional Anti-Cancer Agent

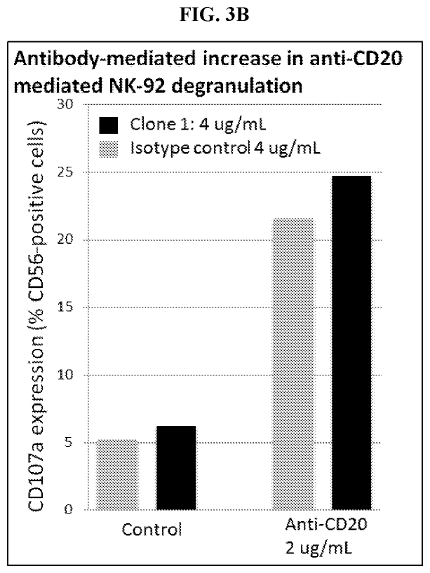

FIG. 3A exemplifies anti-HLA-E-VMAPRTLFL antibody in combination with anti-CD20 or in combination with anti-PD-L1 enhanced natural killer (NK) cells degranulation. JVM2 resuspended at 2.10⁴ cells/well were stimulated with the indicated antibodies for 10 minutes. 1.10⁵ primary NK cells were added to the wells and CD107a-Alexa647 antibody was added directly to the wells. Cells were incubated for 1 h, after which brefeldin A (Sigma) and Golgi-Stop (BD Biosciences) were added and the cells were incubated for an additional 5 h. Cells were stained for surface NK cell markers CD56-PE for 30 min. FIG. 3B exemplifies anti-HLA-E-VMAPRTLFL antibody in combination with anti-CD20 enhancedNK-92 degranulation. EB1 resuspended at 2.10⁴ cells/well were stimulated with the indicated antibodies for 10 minutes. 1.10⁵ NK-92 cells were added to the wells and CD107a-Alexa647 antibody was added directly to the wells. Cells were incubated for 1 h, after which brefeldin A (Sigma) and Golgi-Stop (BD Biosciences) were added and the cells were incubated for an additional 5 h. Cells were stained for surface NK cell markers CD56-PE for 30 min.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating cancer characterized by expression of CD94/NKG2A inhibitory receptor in an individual in need thereof, comprising administering to the individual an antibody that selectively binds to a complex comprising an HLA-E and a neoantigen.

2. The method of claim 1, further comprising administering an additional anti-cancer agent.

3. The method of claim 1, further comprising assaying for expression of CD94/NKG2A inhibitory receptor in the individual.

4. The method of claim 1, wherein the cancer is characterized by the overexpression of the CD94/NKG2A inhibitory receptor.

5. The method of claim 1, wherein the antibody does not have a binding affinity to (i) the HLA-E alone; or (ii) the neoantigen alone.

6. The method of claim 1, wherein the neoantigen comprises, consists essentially of, or consists of a sequence VMAPRTLFL.

7. The method of claim 1, wherein the HLA-E is HLA-E*0101 or HLA-E*0103.

8. The method of claim 7, wherein the antibody selectively binds to the complex comprising:
(a) the HLA-E*0101 and the neoantigen;
(b) the HLA-E*0103 and the neoantigen; or
(c) the HLA-E*0101 and the neoantigen, and the HLA-E*0103 and the neoantigen.

9. The method of claim 1, wherein the complex comprises the HLA-E and VMAPRTLFL.

10. The method of claim 1, wherein the antibody is a murine antibody, a chimeric antibody, a camelid antibody, a humanized antibody, or a human antibody.

11. The method of claim 1, wherein the antibody is a TCR-like antibody.

12. The method of claim 1, wherein the antibody is a single domain antibody.

13. The method of claim 12, wherein the single domain antibody is a camelid single domain antibody.

14. The method of claim 1, wherein the antibody is a multispecific antibody.

15. The method of claim 1, wherein the antibody is a multifunctional antibody.

16. The method of claim 1, wherein the selective binding of the antibody to the complex comprising the HLA-E and the neoantigen inhibits the binding of the complex to the CD94/NKG2A inhibitory receptor.

17. The method of claim 16, wherein the inhibition in binding of the complex to the CD94/NKG2A inhibitory receptor induces activation of natural killer (NK) cells.

18. The method of claim 16, wherein the inhibition in binding of the complex to the CD94/NKG2A inhibitory receptor induces activation of CD8+ T cells.

19. The method of claim 16, wherein the inhibition in binding of the complex to the CD94/NKG2A inhibitory receptor induces cell death of a cell expressing the HLA-E and the neoantigen.

20. The method of claim 19, wherein the cell is a cancer cell.

21. The method of claim 2, wherein the additional anti-cancer agent comprises CD20 inhibitor, HER-2 inhibitor, CD52 inhibitor, EGFR inhibitor, VEGF inhibitor, CCR4 inhibitor, PD-L1 inhibitor, SLAMF7 inhibitor, CD38 inhibitor, GD2 inhibitor, PTK-7 inhibitor, P-cadherin inhibitor, MCSF inhibitor, IDO1 inhibitor, CCR2 inhibitor, CXCR4 inhibitor, PD-1 inhibitor, CTLA-4 inhibitor, OX40 agonist, 4-1BB agonist, androgen receptor inhibitor, tyrosine kinase inhibitor, PARP inhibitor, chimeric antigen receptor T cells (CAR-T cells), oncolytic virus, and any combination thereof.

22. The method of claim 2, wherein the additional anti-cancer agent comprises Rituximab, Trastuzumab, Alemutuzumab, Cetixumab, Bevacizumab, Panitumumab, Obinutuzumab, Mogamulizumab, Necitumumab, Atezolizumab, Elotuzumab, Daratumumab, Dinutuximab, and any combination thereof.

23. The method of claim 2, wherein the antibody and the additional anti-cancer agent are administered concurrently.

24. The method of claim 2, wherein the antibody and the additional anti-cancer agent are administered sequentially.

25. The method of claim 2, wherein the antibody is administered prior to the additional anti-cancer agent.

26. The method of claim 2, wherein the antibody is administered after the additional anti-cancer agent.

27. The method of claim 2, wherein the antibody and the additional anti-cancer agent are administered in a unified dosage form.

28. The method of claim 2, wherein the antibody and the additional anti-cancer agent are administered in a separate dosage form.

29. The method of claim 2, wherein the antibody and the additional anti-cancer agent are administered continuously for 1, 2, 3, 4, 5, 6, 7, 8, 9, 10, 14, 15, 28, 30 or more days.

30. The method of claim 2, wherein the antibody and the additional anti-cancer agent are administered at predetermined time intervals for 1, 2, 3, 4, 5, 6, 7, 8, 9, 10, 14, 15, 28, 30 or more days.

31. The method of claim 2, wherein the antibody and the additional anti-cancer agent are administered intermittently for 1, 2, 3, 4, 5, 6, 7, 8, 9, 10, 14, 15, 28, 30 or more days.

32. The method of claim 2, wherein the antibody and the additional anti-cancer agent are administered in 1 dose, 2 doses, 3 doses, 4 doses, 5 doses, 6 doses or more.

33. The method of claim 2, wherein the antibody and the additional anti-cancer agent are administered at a therapeutically effective amount.

34. The method of claim 1, wherein the cancer is breast cancer, kidney cancer, lung cancer, ovarian cancer, or colorectal cancer.

35. The method of claim 1, wherein the cancer is a B-cell malignancy.

36. A method of treating cancer in an individual in need thereof, comprising administering to the individual:
(a) an antibody that selectively binds to a complex comprising a HLA-E and a neoantigen, and
(b) an additional anti-cancer agent.

37. The method of claim 36, wherein the antibody does not have a binding affinity to (i) the HLA-E alone; or (ii) the neoantigen alone.

38. The method of claim 36, wherein the neoantigen comprises, consists essentially of, or consists of a sequence VMAPRTLFL.

39. The method of claim 36, wherein the HLA-E is HLA-E*0101 or HLA-E*0103.

40. The method of claim 39, wherein the antibody selectively binds to the complex comprising:
(a) the HLA-E*0101 and the neoantigen;
(b) the HLA-E*0103 and the neoantigen; or
(c) the HLA-E*0101 and the neoantigen, and the HLA-E*0103 and the neoantigen.

41. The method of claim 36, wherein the complex comprises the HLA-E and VMAPRTLFL.

42. The method of claim 36, wherein the antibody is a murine antibody, a chimeric antibody, a camelid antibody, a humanized antibody, or a human antibody.

43. The method of claim 36, wherein the antibody is a TCR-like antibody.

44. The method of claim 36, wherein the antibody is a single domain antibody.

45. The method of claim 44, wherein the single domain antibody is a camelid single domain antibody.

46. The method of claim 36, wherein the antibody is a multispecific antibody.

47. The method of claim 36, wherein the antibody is a multifunctional antibody.

48. The method of claim 36, wherein the selective binding of the antibody to the complex comprising the HLA-E and the neoantigen inhibits the binding of the complex to a CD94/NKG2A inhibitory receptor.

49. The method of claim 48, wherein the inhibition in binding of the complex to the CD94/NKG2A inhibitory receptor induces activation of natural killer (NK) cells.

50. The method of claim 48, wherein the inhibition in binding of the complex to the CD94/NKG2A inhibitory receptor induces activation of CD8+ T cells.

51. The method of claim 48, wherein the inhibition in binding of the complex to the CD94/NKG2A inhibitory receptor induces cell death of a cell expressing the HLA-E and the neoantigen.

52. The method of claim 51, wherein the cell is a cancer cell.

53. The method of claim 36, wherein the additional anti-cancer agent comprises CD20 inhibitor, HER-2 inhibitor, CD52 inhibitor, EGFR inhibitor, VEGF inhibitor, CCR4 inhibitor, PD-L1 inhibitor, SLAMF7 inhibitor, CD38 inhibitor, GD2 inhibitor, PTK-7 inhibitor, P-cadherin inhibitor, MCSF inhibitor, IDO1 inhibitor, CCR2 inhibitor, CXCR4 inhibitor, PD-1 inhibitor, CTLA-4 inhibitor, OX40 agonist, 4-1BB agonist, androgen receptor inhibitor, tyrosine kinase inhibitor, PARP inhibitor, chimeric antigen receptor T cells (CAR-T cells), oncolytic virus, and any combination thereof.

54. The method of claim 36, wherein the additional anti-cancer agent comprises Rituximab, Trastuzumab, Alemutuzumab, Cetixumab, Bevacizumab, Panitumumab, Obinutuzumab, Mogamulizumab, Necitumumab, Atezolizumab, Elotuzumab, Daratumumab, Dinutuximab, and any combination thereof.

55. The method of claim 36, wherein the antibody and the additional anti-cancer agent are administered concurrently.

56. The method of claim 36, wherein the antibody and the additional anti-cancer agent are administered sequentially.

57. The method of claim 36, wherein the antibody is administered prior to the additional anti-cancer agent.

58. The method of claim 36, wherein the antibody is administered after the additional anti-cancer agent.

59. The method of claim 36, wherein the antibody and the additional anti-cancer agent are administered in a unified dosage form.

60. The method of claim 36, wherein the antibody and the additional anti-cancer agent are administered in a separate dosage form.

61. The method of claim 36, wherein the antibody and the additional anti-cancer agent are administered continuously for 1, 2, 3, 4, 5, 6, 7, 8, 9, 10, 14, 15, 28, 30 or more days.

62. The method of claim 36, wherein the antibody and the additional anti-cancer agent are administered at predetermined time intervals for 1, 2, 3, 4, 5, 6, 7, 8, 9, 10, 14, 15, 28, 30 or more days.

63. The method of claim 36, wherein the antibody and the additional anti-cancer agent are administered intermittently for 1, 2, 3, 4, 5, 6, 7, 8, 9, 10, 14, 15, 28, 30 or more days.

64. The method of claim 36, wherein the antibody and the additional anti-cancer agent are administered in 1 dose, 2 doses, 3 doses, 4 doses, 5 doses, 6 doses or more.

65. The method of claim 36, wherein the antibody and the additional anti-cancer agent are administered at a therapeutically effective amount.

66. The method of claim 36, wherein the cancer is characterized by expression of the CD94/NKG2A inhibitory receptor.

67. The method of claim 36, wherein the cancer is characterized by overexpression of the CD94/NKG2A inhibitory receptor.

68. The method of claim 66, further comprising assaying for expression of the CD94/NKG2A inhibitory receptor in the individual.

69. The method of claim 36, wherein the cancer is breast cancer, kidney cancer, lung cancer, ovarian cancer, or colorectal cancer.

70. The method of claim 36, wherein the cancer is a B-cell malignancy.

71. A method of inducing cell death of a cancer cell in a tumor microenvironment characterized by expression of CD94/NKG2A inhibitory receptor in an individual in need thereof, comprising administering to the individual an antibody that selectively binds to a complex comprising an HLA-E and a neoantigen, wherein the complex is expressed by the cancer cell.

72. The method of claim 71, further comprising administering an additional anti-cancer agent.

73. The method of claim 71, further comprising assaying for expression of the CD94/NKG2A inhibitory receptor in the individual.

74. The method of claim 71, wherein the tumor microenvironment is characterized by the overexpression of the CD94/NKG2A inhibitory receptor.

75. The method of claim 71, wherein the antibody does not have a binding affinity to (i) the HLA-E alone; or (ii) the neoantigen alone.

76. The method of claim 71, wherein the neoantigen comprises, consists essentially of, or consists of a sequence VMAPRTLFL.

77. The method of claim 71, wherein the HLA-E is HLA-E*0101 or HLA-E*0103.

78. The method of claim 77, wherein the antibody selectively binds to the complex comprising:
(a) the HLA-E*0101 and the neoantigen;
(b) the HLA-E*0103 and the neoantigen; or
(c) the HLA-E*0101 and the neoantigen, and the HLA-E*0103 and the neoantigen.

79. The method of claim 71, wherein the complex comprises the HLA-E and VMAPRTLFL.

80. The method of claim 71, wherein the antibody is a murine antibody, a chimeric antibody, a camelid antibody, a humanized antibody, or a human antibody.

81. The method of claim 71, wherein the antibody is a TCR-like antibody.

82. The method of claim 71, wherein the antibody is a single domain antibody.

83. The method of claim 82, wherein the single domain antibody is a camelid single domain antibody.

84. The method of claim 71, wherein the antibody is a multispecific antibody.

85. The method of claim 71, wherein the antibody is a multifunctional antibody.

86. The method of claim 71, wherein the selective binding of the antibody to the complex comprising the HLA-E and the neoantigen inhibits the binding of the complex to the CD94/NKG2A inhibitory receptor.

87. The method of claim 86, wherein the inhibition in binding of the complex to the CD94/NKG2A inhibitory receptor induces activation of natural killer (NK) cells.

88. The method of claim 86, wherein the inhibition in binding of the complex to the CD94/NKG2A inhibitory receptor induces activation of CD8+ T cells.

89. The method of claim 72, wherein the additional anti-cancer agent comprises CD20 inhibitor, HER-2 inhibitor, CD52 inhibitor, EGFR inhibitor, VEGF inhibitor, CCR4 inhibitor, PD-L1 inhibitor, SLAMF7 inhibitor, CD38 inhibitor, GD2 inhibitor, PTK-7 inhibitor, P-cadherin inhibitor, MCSF inhibitor, IDO1 inhibitor, CCR2 inhibitor, CXCR4 inhibitor, PD-1 inhibitor, CTLA-4 inhibitor, OX40 agonist, 4-1BB agonist, androgen receptor inhibitor, tyrosine kinase inhibitor, PARP inhibitor, chimeric antigen receptor T cells (CAR-T cells), oncolytic virus, and any combination thereof.

90. The method of claim 72, wherein the additional anti-cancer agent comprises Rituximab, Trastuzumab, Alemutuzumab, Cetixumab, Bevacizumab, Panitumumab, Obinutuzumab, Mogamulizumab, Necitumumab, Atezolizumab, Elotuzumab, Daratumumab, Dinutuximab, and any combination thereof.

91. The method of claim 72, wherein the antibody and the additional anti-cancer agent are administered concurrently.

92. The method of claim 72, wherein the antibody and the additional anti-cancer agent are administered sequentially.

93. The method of claim 72, wherein the antibody is administered prior to the additional anti-cancer agent.

94. The method of claim 72, wherein the antibody is administered after the additional anti-cancer agent.

95. The method of claim 72, wherein the antibody and the additional anti-cancer agent are administered in a unified dosage form.

96. The method of claim 72, wherein the antibody and the additional anti-cancer agent are administered in a separate dosage form.

97. The method of claim 72, wherein the antibody and the additional anti-cancer agent are administered continuously for 1, 2, 3, 4, 5, 6, 7, 8, 9, 10, 14, 15, 28, 30 or more days.

98. The method of claim 72, wherein the antibody and the additional anti-cancer agent are administered at predetermined time intervals for 1, 2, 3, 4, 5, 6, 7, 8, 9, 10, 14, 15, 28, 30 or more days.

99. The method of claim 72, wherein the antibody and the additional anti-cancer agent are administered intermittently for 1, 2, 3, 4, 5, 6, 7, 8, 9, 10, 14, 15, 28, 30 or more days.

100. The method of claim 72, wherein the antibody and the additional anti-cancer agent are administered in 1 dose, 2 doses, 3 doses, 4 doses, 5 doses, 6 doses or more.

101. The method of claim 72, wherein the antibody and the additional anti-cancer agent are administered at a therapeutically effective amount.

102. The method of claim 71, wherein the cancer cell is a breast cancer cell, a kidney cancer cell, a lung cancer cell, an ovarian cancer cell, or a colorectal cancer cell.

103. The method of claim 71, wherein the cancer cell is a malignant B cell.

104. A pharmaceutical composition comprising:
(a) an antibody that selectively binds to a complex comprising a HLA-E and a neoantigen;
(b) an additional anti-cancer agent; and
(c) a pharmaceutically acceptable carrier or excipient.

105. The pharmaceutical composition of claim 104 for use in treating a cancer.

106. The pharmaceutical composition of claim 104 for use in treating a cancer characterized by expression of CD94/NKG2A inhibitory receptor.

107. The pharmaceutical composition of claim 104 for use in treating a cancer characterized by overexpression of CD94/NKG2A inhibitory receptor.
